(19) United States
(12) Patent Application Publication
(10) Pub. No.: US 2007/0190229 A1
(43) Pub. Date: Aug. 16, 2007

(54) Medical adhesive and methods of tissue adhesion

(76) Inventors: Eric J. Beckman, Aspinwall, PA (US); Jianying Zhang, Pittsburgh, PA (US)

Correspondence Address: BARTONY & HARE, LLP, 1806 FRICK BUILDING, 437 GRANT STREET, PITTSBURGH, PA, 15219-6101, US

(21) Appl. No.: 11/698,353

(22) Filed: Jan. 26, 2007

Related U.S. Application Data
(60) Provisional application No. 60/762,634, filed on Jan. 27, 2006.

(51) Int. Cl. A61L 33/00; B05D 3/00

(52) U.S. Cl. 427/2.1; 524/589; 528/44

(57) ABSTRACT

A method of adhering biological tissue that includes applying a bio-degradable adhesive to the tissue. The adhesive includes a moisture-curable, isocyanate-functional component prepared by reacting (a) a multi-functional isocyanate component and (b) a multi-functional active hydrogen component that includes at least 30% by weight, based upon the total weight of the multi-functional active hydrogen component, of a multi-functional active hydrogen reactant having an equivalent weight less than 100. The ratio R of active hydrogen groups to isocyanate groups can be less than 1.0.

### CROSS-REFERENCE TO RELATED APPLICATIONS

This application derives priority from U.S. patent application Ser. No. 60/762,634, entitled MEDICAL ADHESIVE AND METHODS OF TISSUE ADHESION, filed Jan. 27, 2006, the entire disclosure of which is incorporated herein by reference.

### TECHNICAL FIELD

This invention relates to medical adhesives and to methods of tissue adhesion.

### BACKGROUND

Each year approximately eleven million traumatic wounds are treated by emergency physicians in the United States. Traumatic wounds rival respiratory tract infections as the most common reason people seek medical care. Conventional methods of tissue closure (for example, sutures and staples) have several substantial limitations, including inability to produce fluid-tight closure, unsuitability for microsurgical applications, necessity for a second operation for removal, increased probability of inflammation and infection, and significant scarring and tissue injury during insertion. Medical tapes have been used for some applications, but medical tapes are limited by weak strength and problems with adherence to tissue. Treatment of lacerations with sutures often involves the injection of local anesthetic and use of needles, which can distress an already frightened patient. See, for example, McCraig L F, “National Hospital Ambulatory Medical Care Survey: 1992 Emergency Department Summary, Vital Health Stat., 1994, 245, 1-12; and Eland J M, Anderson J E, “The Experience of Pain in Children,” in Jacos A K, ed. Pain, Boston, Mass.: Little Brown & Co., 1997, 453-473. Suture wound repair is also painful and time-consuming. For quite some time, physicians have sought wound repair methods that require little time, do not require additional surgery, minimize the discomfort to their patients, and produce a good cosmetic outcome.

In an attempt to achieve such goals, both biological and synthetic tissue adhesives have been developed. Examples of known biological tissue adhesives include fibrin glues. Examples of known synthetic tissue adhesives include cyanoacrylates, urethane prepolymers, and gelatin-resorcinol-formaldehyde. Applications of adhesives to biological tissue range from soft (connective) tissue adhesion to hard (calcified) tissue adhesion. Soft tissue adhesives are, for example, used both externally and internally for wound closure and sealing. Hard tissue adhesives are used, for example, to bond prosthetic materials to teeth and bone.

### SUMMARY

A method of adhering biological tissue is described that includes applying a bio-degradable adhesive to the tissue. The adhesive includes a moisture-curable, isocyanate-functional component prepared by reacting (a) a multi-functional isocyanate component and (b) a multi-functional active hydrogen component that includes at least 30% by weight, based upon the total weight of the multi-functional active hydrogen component, of a multi-functional active hydrogen reactant having an equivalent weight less than 100. The ratio R of active hydrogen groups to isocyanate groups in preparing the isocyanate functional component can be less than 1.0. Upon application to tissue, and in the presence of moisture, the compositions crosslink (i.e., cure) to form a polymer network.

The term “component” refers to single compounds, and to blends of different compounds. Thus, for example, a “moisture-curable, isocyanate-functional component” (which refers to that portion of the adhesive prepared by reacting a multi-functional isocyanate component and a multi-functional active hydrogen component) can include one moisture-curable, isocyanate-functional prepolymer, or a blend of moisture-curable, isocyanate-functional prepolymers having different compositions. Similarly, a “multi-functional isocyanate component” can include a single multi-functional isocyanate compound, or a blend of different multi-functional isocyanate compounds. In the case of blends of isocyanate-functional prepolymers, R can be greater than 1 or less than 1 for individual prepolymers, but R can be less than 1 for the resultant isocyanate-functional component. Likewise, the “multi-functional active hydrogen component” can include only a multi-functional active hydrogen reactant having an equivalent weight less than 100, or can include blends of this reactant with (a) other multi-functional active hydrogen reactants having an equivalent weight less than 100 but a different chemical composition and/or (b) one or more multi-functional active hydrogen reactants that have equivalent weights greater than 100.

The term “equivalent weight” refers to molecular weight divided by functionality. Thus, for example, glycerol, which has a molecular weight of 92 and a hydroxyl functionality “f” of 3, has an equivalent weight of approximately 31. Glucose, which has a molecular weight of 180 and a functionality “f” of 5, has an equivalent weight of 36.

Certain embodiments of the moisture-curable, isocyanate-functional composition (i.e., those in which f>2 and h=2) alternatively may be defined in terms of its chain length, designated “Xn,” calculated according to the following equation:
Xn=(frp+1−rp)/(1−rp)
where “f” is the average functionality of the multi-functional active hydrogen reactant, “r” is the ratio of the total number of active hydrogen groups to the total number of isocyanate groups, and “p” represents the extent of reaction and is equal to one. In several embodiments, the compositions have values of Xn greater than 4 but no greater than 61. For example, Xn may be in the range of 7 to 61, 7 to 41, or 7 to 22.

A biodegradable moisture-curable, isocyanate-functional composition is also described that includes the reaction product of (a) a multi-functional isocyanate component and (b) a multi-functional active hydrogen component including at least 30% by weight, based upon the total weight of the multi-functional active hydrogen component, of a multi-functional active hydrogen reactant having an equivalent weight less than 100. The composition also includes an agent selected from the group consisting of catalysts, latent hardening agents, rheology modifying agents, and combinations thereof.

Further, a composition is described that is prepared by reacting (a) a multi-functional isocyanate component having an average functionality of h and (b) a multi-functional active hydrogen component having an average functionality of at least f and consisting essentially of multi-functional active hydrogen reactants having an equivalent weight less than 100, wherein a ratio R of active hydrogen groups to isocyanate groups is selected such that 1/h<R<0.9.

The adhesive and other compositions are readily synthesized and provide a minimally invasive avenue to applications such as tissue closure. The modulus or stiffness of the compositions may be adjusted for use, for example, either as soft/flexible (connective) tissue adhesives (e.g., skin adhesives to replace sutures and staples for closure of certain lacerations and/or incisions) or hard/stiff (calcified) tissue adhesives (e.g., bone or dental adhesives) in humans and animals.

The details of one or more embodiments are set forth in the description below. Other features, objects, and advantages of the invention will be apparent from the description, and from the claims.

### DETAILED DESCRIPTION

In several embodiments, bio-degradable adhesives suitable for application to biological tissue (for example, soft tissue) include a moisture-curable, isocyanate-functional component prepared by reacting (a) a multi-functional isocyanate component and (b) a multi-functional active hydrogen component that includes at least 30% by weight, based upon the total weight of the multi-functional active hydrogen component, of a multi-functional active hydrogen reactant having an equivalent weight less than 100.

The ratio R of active hydrogen groups to isocyanate groups can be less than 1.0. In some embodiments, R is selected such that 0.5≦R<0.9. For example, in some embodiments, multi-functional isocyanate component has an average functionality of 2, the multi-functional active hydrogen component has an average functionality of at least 3, and R is selected such that 0.5<R<0.9; 0.5<R<0.8; or 0.5<R<0.67. In other embodiments, the multi-functional isocyanate component has an average functionality of 3, the multi-functional active hydrogen component has an average functionality of at least 2, and R is selected such that 0.33<R<0.9; 0.33<R<0.8; or 0.33<R, 0.67. Alternatively, the composition may be described in terms of its chain length “Xn,” defined in the Summary, above.

Upon application to biological tissue in the presence of moisture, the adhesive crosslinks to form a polymer network. To form the network, the moisture-curable, isocyanate-functional component of the adhesive has an average isocyanate functionality of greater than 2, and preferably greater than 2.1. Typically, the isocyanate functionality is at least 2.5 or at least 3. The term “average” reflects the fact that the moisture-curable, isocyanate-functional component, as explained in the Summary, above, can include multiple moisture-curable, isocyanate-functional prepolymers having different chemical compositions. In that regard, functionality (and other characteristics) can be determined on the basis of molar averages.

The crosslinked network biodegrades over time. For example, it can biodegrade in a period of time during which healing occurs. It can, for example, remain intact to adhere the tissue of a laceration or an incision until healing has sufficiently progressed such that the wound or incision remains closed. This can occur over a period of days or months, for example, depending upon the adhesive. In one embodiment, the crosslinked network biodegrades to lose at least approximately ⅔ of its material in approximately 3 to approximately 60 days.

The multi-functional isocyanate component has an average isocyanate functionality of at least 2. In several embodiments, the average isocyanate functionality is 2, while in several other embodiments it is 3. The term “average” reflects the fact that the multi-functional isocyanate component, as explained in the Summary, above, can include multiple types of multi-functional isocyanates. Suitable multi-functional isocyanates include hydrophilic multi-functional isocyanates, and include those derived from amino acids and amino acid derivatives. Specific examples include lysine di-isocyanate (“LDI”) and derivatives thereof (e.g., alkyl esters such as methyl or ethyl esters) and lysine tri-isocyanate (“LTI”) and derivatives thereof (e.g., alkyl esters such as methyl or ethyl esters). Dipeptide derivatives can also be used. For example, lysine can be combined in a dipeptide with another amino acid (e.g., valine or glycine). In addition, isocyanates prepared from putrescine (diamino butane) can be used as well. One class of suitable multi-isocyanates includes generally those multi-isocyanate derived from biocompatible multi-functional amines. As used herein, the term “biocompatible” refers generally to compatibility with living tissue or a living system.

The multi-functional active hydrogen component includes one or more multi-functional active hydrogen reactants. The component has an average functionality of at least 2, and may be 3 or more. Again, the term “average” reflects the fact that the multi-functional active hydrogen component, as explained in the Summary, above, can include multiple types of multi-functional active hydrogen reactants.

The multi-functional active hydrogen component contains at least 30% by weight, based upon the total weight of the multi-functional active hydrogen component, of a multi-functional active hydrogen reactant having an equivalent weight less than 100. In some embodiments, the equivalent weight is less than 50, while in other embodiments it is less than 40. In some embodiments, the percentage may be at least 50% or at least 75%, while in other embodiments the multi-functional active hydrogen reactant consists essentially of multi-functional active hydrogen reactants having an equivalent weight less than 100 (or less than 50; or less than 40). In other embodiments, the multi-functional active hydrogen-containing component is free of multi-functional active hydrogen reactants having main chain ether or ester linkages. A “main chain ether or ester linkage” is a linkage that appears in the backbone of the molecule, as opposed to a side group or side chain. In several embodiments, multi-functional active hydrogen reactants of the multi-functional active hydrogen component can have a molecular less than 600, less than 400 or less than 200

Suitable multi-functional active hydrogen reactants include polyols, polyamines, and polythiols. One class of suitable polyols having equivalent weights less than 100 includes glycerol, di-glycerol, pentaerythritol, xylitol, arabitol, fucitol, ribitol, sorbitol, mannitol, and combinations thereof. Another class of suitable polyols having equivalent weights less than 100 includes saccharides (e.g., glucose, fructose, sucrose, and lactose), oligosaccharides polysaccharides, and combinations thereof. Also useful are polyols having equivalent weights less than 100 selected from steroids, ascorbic acid, gluconic acid, glucoronic acid, glycosamine, and combinations thereof.

Another class of suitable multi-functional active hydrogen reactants includes generally biocompatible multi-functional active hydrogen reactants having equivalent weights less than 100.

The multi-functional active hydrogen-containing composition can also include multi-functional active hydrogen reactants having an equivalent weight greater than 100, subject to the above-described weight percentage limitations set forth in the Summary of the Invention. Examples of representative reactants include polyesters, polyethers, polyalkylene oxides, polyamino acids, polycarbonates, polyanhydrides, and the like, which include multiple active hydrogen groups.

One example of a bio-degradable adhesive suitable for application to biological tissue includes a moisture-curable, isocyanate-functional component prepared by reacting: (a) a multi-functional isocyanate component having an average functionality of 2; and (b) a multi-functional active hydrogen component having an average functionality of at least 3 that includes at least 30% by weight, based upon the total weight of the multi-functional active hydrogen component, of a multi-functional active hydrogen reactant having an equivalent weight less than 100. The ratio R of active hydrogen groups to isocyanate groups is selected such that 0.5<R<0.9. In addition, the multi-functional active hydrogen component is free of multi-functional active hydrogen reactants having main chain ether or ester linkages.

Another example of a bio-degradable adhesive suitable for application to biological tissue includes a moisture-curable, isocyanate-functional component prepared by reacting: (a) a multi-functional isocyanate component having an average functionality of 3; and (b) a multi-functional active hydrogen component having an average functionality of at least 2 that includes at least 30% by weight, based upon the total weight of the multi-functional active hydrogen component, of a multi-functional active hydrogen reactant having an equivalent weight less than 100. The ratio R of active hydrogen groups to isocyanate groups is selected such that 0.33<R<0.9. In addition, the multi-functional active hydrogen component is free of multi-functional active hydrogen reactants having main chain ether or ester linkages.

The moisture-curable, isocyanate-functional component can be formed by reacting one or more multi-functional isocyanate reactants and one or more multi-functional active hydrogen components, either sequentially or in a one-pot reaction. Alternatively, multiple moisture-curable, isocyanate-functional prepolymers can be prepared separately and then blended together to form the moisture-curable, isocyanate-functional component.

The adhesive may further include one or more agents selected from catalysts, latent hardening agents, rheology modifying agents, and combinations thereof. Examples of suitable catalysts include tertiary amines (e.g., aliphatic tertiary amines) and organometallic compounds. Specific examples include 1,4-diazabicyclo[2.2.2]octane (“DABCO”), 2,2′dimorpholine diethyl ether (“DMDEE”), dibutyltin dilaurate (“DBTDL”), bismuth 2-ethylhexanoate, and combinations thereof. The amount of catalyst is selected based upon the particular reactants. In general, however, the amount of catalyst, when present, is no greater than about 5% by weight, based upon the total weight of the adhesive, and preferably no greater than about 2% by weight.

The latent hardening agent may be used to adjust the “open time” of the adhesive (i.e., the amount of time before it crosslinks and becomes a thermoset material). The “open time,” in turn, is selected based upon the needs of the particular application for which the adhesive is being used. In general, it may range from approximately 30 seconds to approximately 10 minutes, more typically from approximately 30 seconds to approximately 5 minutes, and even more typically from approximately 3 minutes to approximately 5 minutes.

Suitable examples of latent hardening agents include multi-functional imines, e.g., synthesized from biocompatible aldehydes (e.g., 4-hydroxy-3-methoxybenzaldehyde) and biocompatible multi-functional amines (e.g., amino acids or derivatives thereof, including lysine and lysine esters). The amount of latent hardening agent is selected based upon the constituents of the adhesive and the desired “open time.” The latter, in turn, may depend upon the particular application for which the adhesive is being used. In general, the amount of latent hardening agent, when present, is no greater than about 30% by weight, based upon the total weight of the adhesive. In some embodiments, the amount is no greater than 15% by weight, while in others the amount is no greater than 10% by weight.

The rheology modifying agent is used to modify the rheology of the adhesive (including its viscosity) to achieve desired handling characteristics for a particular application. In general, the viscosity of the adhesive is in the range of about 1 to 170,000 centipoise (measured at 20° C.), and more preferably in the range of about 1 to 150,000 centipoise or 1 to 100,000 to facilitate application of the adhesive. To create an adhesive that is sprayable or injectable, the viscosity preferably is in the range of about 1 to 5,000 centipoise, preferably 1 to 2,000 centipoise. Adhesives designed to be spread on a site preferably have a viscosity in the range of about 100 to 150,000 centipoise, preferably about 5,000 to 50,000 centipoise.

Useful rheology modifying agents include materials that act as solvents for the adhesive. Specific examples include triacetin, dimethyl isosorbide, soy ethyl ester, dimethyl sulfoxide (“DMSO”), propylene carbonate, and glymes. In addition, excess multi-functional isocyanate (e.g., excess LDI and/or LTI) can also perform the role of a rheology modifying agent. The amount of the rheology modifying agent is selected based upon the constituents of the adhesive and the particular application for which the adhesive is being used. In general, the amount of rheology modifying agent, when present, is no greater than about 70% by weight, based upon the total weight of the adhesive. In several embodiments, the rheology modifying agent does not react with isocyanate functional groups.

### EXAMPLES

### Example 1

### LDI/Glycerol (R=0.64) plus 1,4-diazabicyclo[2.2.2]octane (DABCO

An LDI-based polyurethane tissue adhesive was synthesized by the following procedure using DABCO as a catalyst. In this procedure, 0.10 g DABCO and 1.57 g glycerol (17.0 mmol, —OH 51.0 mmol) was added to 8.43 g of LDI (39.7 mmol, —NCO 79.5 mmol) in a dry 20 mL beaker. The reaction mixture was stirred at room temperature for approximately one hour, and a viscous liquid was obtained. The viscous liquid was kept at room temperature under nitrogen until use. The viscous liquid was spread onto each of two pieces of fresh bovine muscle tissue, which when pressed together adhered firmly to each other after 1-3 minutes.

### Example 2

### LDI/Glycerol (R=0.67) plus DABCO

Another LDI-based polyurethane tissue adhesive was synthesized by the following procedure. 0.10 g DABCO and 1.62 g glycerol (17.6 mmol, —OH 52.9 mmol) was added to 8.38 g of LDI (39.5 mmol, —NCO 79.0 mmol) in a dry 20 mL beaker. The reaction mixture was stirred at room temperature for approximately one hour, and a viscous liquid was obtained. The viscous liquid was kept at room temperature under nitrogen until use. The viscous liquid was spread onto each of two pieces of fresh bovine muscle tissue, which when pressed together would adhere firmly to each other after approximately 1-3 minutes.

### Example 3

### LDI/Glycerol/DABCO (R=0.67) plus additional LDI

Another LDI-based polyurethane tissue adhesive was synthesized by the following procedure. 0.10 g DABCO and 1.62 g glycerol (17.6 mmol, —OH 52.9 mmol) was added to 8.38 g of LDI (39.5 mmol, —NCO 79.0 mmol) in a dry 20 mL beaker. The reaction mixture was stirred at room temperature for approximately an hour, and a viscous solution was obtained. At this point 8.38 g LDI (39.5 mmol, —NCO 79.0 mmol) was added to the reaction mixture. The reaction product was stirred for 5 minutes, and produced a highly viscous fluid. The viscous liquid was kept at room temperature under nitrogen.

### Example 4

### LDI/Glycerol (R=0.67) plus 2,2′dimorpholine diethyl ether (DMDEE)

Another LDI-based polyurethane tissue adhesive was synthesized by the following procedure. 0.10 g DMDEE and 1.62 g glycerol (17.6 mmol, —OH 52.9 mmol) was added to 8.38 g of LDI (39.5 mmol, —NCO 79.0 mmol) in a dry 20 mL beaker. The reaction mixture was stirred at room temperature overnight, and a viscous liquid was obtained. The viscous liquid was kept at room temperature under nitrogen until use. The viscous liquid was spread onto each of two pieces of fresh bovine muscle tissue, which when pressed together would adhere firmly to each other after approximately 1-3 minutes.

### Example 5

### LDI/Glycerol/dibutyltin dilaurate (DBTDL) (R=0.64) plus DMDEE

Another LDI-based polyurethane tissue adhesive was synthesized by the following procedure. 9.4 μL (0.10% by weight) DBTDL and 1.57 g glycerol (17.0 mmol, —OH 51.0 mmol) was added to 8.43 g of LDI (39.7 mmol, —NCO 79.5 mmol) in a dry 20 mL beaker. The reaction mixture was stirred at room temperature for approximately 30 minutes, and a viscous liquid was obtained. At this point 0.10 g DMDEE was added to the reaction mixture. The viscous liquid was kept at room temperature under nitrogen until use. The viscous liquid was spread onto each of two pieces of fresh bovine muscle tissue, which when pressed together would adhere firmly to each other after approximately 1-3 minutes.

### Example 6

### LDI/Glycerol/DBTDL (R=0.67) in dimethyl formamide (DMF) plus DMDEE

Another LDI-based polyurethane tissue adhesive was synthesized by the following procedure. 1.62 g glycerol (17.6 mmol, —OH 52.9 mmol) and 8.38 g of LDI (39.5 mmol, —NCO 79.0 mmol) were added to 5.0 g DMF in a dry 20 mL beaker and mixed until homogeneous. 9.4 μL (0.10% by weight) DBTDL was added. The reaction mixture was stirred at room temperature for approximately 10 minutes, and a viscous liquid was obtained. At this point 0.30 g DMDEE was added to the reaction mixture. The viscous liquid was kept at room temperature under nitrogen until use. The viscous liquid was spread onto each of two pieces of fresh bovine muscle tissue, which when pressed together would adhere firmly to each other after approximately 1-3 minutes.

### Example 7

### LDI/Glycerol/bismuth 2-ethylhexanoate (R=0.67) in dimethyl sulfoxide (DMSO) plus DMDEE

Another LDI-based polyurethane tissue adhesive was synthesized by the following procedure. 1.62 g glycerol (17.6 mmol, —OH 52.9 mmol) and 8.38 g of LDI (39.5 mmol, —NCO 79.0 mmol) were added to 4.5 g DMSO in a dry 20 mL beaker and mixed until homogeneous. 8.0 μL bismuth 2-ethylhexanoate was added. The reaction mixture was stirred at room temperature for approximately 1 minute, and a viscous liquid was obtained. At this point 0.10 g DMDEE was added to the reaction mixture. The viscous liquid was kept at room temperature under nitrogen until use. The viscous liquid was spread onto each of two pieces of fresh bovine muscle tissue, which when pressed together would adhere firmly to each other after approximately 1-3 minutes.

### Example 8

### LDI/Glycerol/DBTDL (R=0.67) in DMF

Another LDI-based polyurethane tissue adhesive was synthesized by the following procedure. 1.26 g glycerol (13.7 mmol, —OH 41.0 mmol) and 8.74 g of LDI (41.2 mmol, —NCO 82.4 mmol) were added to 5.0 g DMF in a dry 20 mL beaker and mixed until homogeneous. 9.4 μL DBTDL was added. The reaction mixture was stirred at room temperature for approximately 10 minutes, and a viscous liquid was obtained. At this point 0.42 g glycerol (4.6 mmol, —OH 13.7 mmol) was added to the reaction mixture to create a chain length 13 product. The reaction product was stirred for 2 minutes, and produced a highly viscous fluid. The viscous liquid was kept at room temperature under nitrogen.

### Example 9

### LDI/Glycerol/bismuth 2-ethylhexanoate (R=0.6) in DMSO

Another LDI-based polyurethane tissue adhesive was synthesized by the following procedure. 1.20 g glycerol (13.0 mmol, —OH 39.1 mmol) was dissolved in 4.54 g of DMSO containing 2 μL of bismuth 2-ethylhexanoate. LDI was added dropwise to the solution to 1.38 g of total LDI (6.51 mmol, —NCO 13.0 mmol). This gives a chain length 3 molecule with R=3. This solution was then added dropwise into a second solution containing 5 g DMSO, 5.53 g LDI (26.03 mmol, —NCO 52.1 mmol), and 4 μL bismuth 2-ethyl hexanoate. The final product creates a viscous fluid with R=0.6. The viscous liquid was kept at room temperature under nitrogen.

### Example 10

### LDI/Glycerol/DBTDL (R=0.67) in p-Dioxane plus bismuth 2-ethylhexanoate and DMDEE and triacetin

Another LDI-based polyurethane tissue adhesive was synthesized by the following procedure. 1.62 g glycerol (17.6 mmol, —OH 52.9 mmol) and 8.38 g of LDI (39.5 mmol, —NCO 79.0 mmol) were added to 27 g of p-dioxane in a dry 20 mL beaker and mixed until homogeneous. 9.4 μL DBTDL was added. The reaction mixture was stirred at room temperature overnight. Dioxane was removed by rotary evaporation, producing a clear viscous fluid. At this point 0.30 g DMDEE and 48 μL of bismuth 2-ethylhexanoate were added to the reaction mixture, as well as 2.0 g (20% by weight) triacetin as a non-reactive diluent. The reaction product was stirred until homogeneous. The viscous liquid was kept at room temperature under nitrogen. The viscous liquid was spread onto each of two pieces of fresh bovine muscle tissue, which when pressed together would adhere firmly to each other after approximately 1-3 minutes.

### Example 11

### LDI/dipentaerythritol (R=0.5) in DMSO

Another LDI-based polyurethane tissue adhesive was synthesized by the following procedure. 1.00 g dipentaerythritol (3.9 mmol, —OH 23.6 mmol) and 5.0 g of LDI (23.6 mmol, —NCO 47.2 mmol) were added to 10.00 g of DMSO and mixed until homogeneous. 0.5 μL bismuth 2-ethylhexanoate was then added. The reaction mixture was stirred in an ice bath for approximately 4 hours, followed by room temperature for 1 hour, resulting in a viscous fluid. The viscous liquid was kept at room temperature under nitrogen.

### Example 12

### LDI/erythritol (R=0.5) in DMSO

Another LDI-based polyurethane tissue adhesive was synthesized by the following procedure. 1.00 g erythritol (8.19 mmol, —OH 32.7 mmol) and 6.95 g of LDI (32.7 mmol, —NCO 65.5 mmol) were added to 8.00 g of DMSO and mixed until homogeneous. 0.5 μL bismuth 2-ethylhexanoate was then added. The reaction mixture was stirred in an ice bath for approximately 1 hour, followed by room temperature for 1 hour, resulting in a viscous fluid. The viscous liquid was kept at room temperature under nitrogen.

### Example 13

### LDI/xylitol (R=0.5) in DMSO

Another LDI-based polyurethane tissue adhesive was synthesized by the following procedure. 1.15 g xylitol (7.6 mmol, —OH 37.8 mmol) and 8.02 g of LDI (37.8 mmol, —NCO 75.6 mmol) were added to 6.25 g of DMSO and mixed until homogeneous. 8 μL bismuth 2-ethylhexanoate was then added. The reaction mixture was stirred in an ice bath for approximately 1 hour, followed by room temperature for 1 hour, resulting in a viscous fluid. The viscous liquid was kept at room temperature under nitrogen.

### Example 14

### LDI/erythritol/glycerol (R=0.5) in DMSO

Another LDI-based polyurethane tissue adhesive was synthesized by the following procedure. 1.0 g erythritol (8.19 mmol, —OH 32.7 mmol), 0.75 g glycerol (8.19 mmol, —OH 24.6 mmol), and 12.16 g of LDI (57.3 mmol, —NCO 114.6 mmol) were added to 10.00 g of DMSO and mixed until homogeneous. 8 μL bismuth 2-ethylhexanoate was then added. The reaction mixture was stirred in an ice bath for approximately 1 hour, followed by room temperature for 1 hour, resulting in a viscous fluid. The viscous liquid was kept at room temperature under nitrogen.

A number of embodiments of the invention have been described. Nevertheless, it will be understood that various modifications may be made without departing from the spirit and scope of the invention. Accordingly, other embodiments are within the scope of the following claims.

What is claimed is:

1. A method of adhering biological tissue comprising applying a bio-degradable adhesive to the tissue,
the adhesive comprising a moisture-curable, isocyanate-functional component prepared by reacting:
(a) a multi-functional isocyanate component; and
(b) a multi-functional active hydrogen component comprising at least 30% by weight, based upon the total weight of the multi-functional active hydrogen component, of a multi-functional active hydrogen reactant having an equivalent weight less than 100.

2. The method of claim 1 wherein the multi-functional active hydrogen component includes at least 50% by weight, based upon the total weight of the component, of a multi-functional active hydrogen reactant having an equivalent weight less than 100.

3. The method of claim 1 wherein the multi-functional active hydrogen component consists essentially of a multi-functional active hydrogen reactant having an equivalent weight less than 100.

4. The method of claim 1 wherein the multi-functional active hydrogen component is selected from the group consisting of hydroxyl-functional components, amino-functional components, and combinations thereof.

5. The method of claim 1 wherein the multi-functional active hydrogen component comprises a hydroxyl-functional component.

6. The method of claim 1 wherein the multi-functional active hydrogen component includes at least 30% by weight, based upon the total weight of the component, of a multi-functional active hydrogen reactant having an equivalent weight less than 50.

7. The method of claim 1 wherein the multi-functional active hydrogen component includes at least 30% by weight, based upon the total weight of the component, of a multi-functional active hydrogen reactant having an equivalent weight less than 40.

8. The method of claim 1 wherein the multi-functional active hydrogen component is free of multi-functional hydrogen reactants having main chain ether or ester linkages.

9. The method of claim 1 wherein the multi-functional active hydrogen component has an average functionality of at least 2.

10. The method of claim 1 wherein the multi-functional active hydrogen component has an average functionality of at least 3.

11. The method of claim 1 wherein the multi-functional isocyanate component has an average functionality of at least 2.

12. The method of claim 1 wherein the multi-functional isocyanate component has an average functionality of at least 3.

13. The method of claim 1 wherein a ratio R of active hydrogen groups to isocyanate groups is selected such that 0.5≦R<0.9.

14. The method of claim 1 wherein the multi-functional isocyanate component has an average functionality of 2, the multi-functional active hydrogen component has an average functionality of at least 3, and a ratio R of active hydrogen groups to isocyanate groups is selected such that 0.5<R<0.9.

15. The method of claim 14 wherein the ratio R is selected such that 0.5<R<0.8.

16. The method of claim 14 wherein the ratio R is selected such that 0.5<R<0.67.

17. The method of claim 1 wherein the multi-functional isocyanate component has an average value of 3, the multi-functional active hydrogen component has an average value of at least 2, and a ratio R of active hydrogen groups to isocyanate groups is selected such that 0.33<R<0.9.

18. The method of claim 17 wherein the ratio R is selected such that 0.33<R<0.8.

19. The method of claim 17 wherein the ratio R is selected such that 0.33<R<0.67.

20. The method of claim 1 wherein the adhesive further comprises an agent selected from the group consisting of catalysts, latent hardening agents, rheology modifying agents, and combinations thereof.

21. The method of claim 1 wherein the multi-functional active hydrogen reactant having an equivalent weight less than 100 is selected from the group consisting of glycerol, di-glycerol, pentaerythritol, xylitol, arabitol, fucitol, ribitol, sorbitol, mannitol, and combinations thereof.

22. The method of claim 1 wherein the multi-functional active hydrogen reactant having an equivalent weight less than 100 is glycerol.

23. The method of claim 1 wherein the multi-functional active hydrogen reactant having an equivalent weight less than 100 is selected from the group consisting of saccharides, oligosaccharides, polysaccharides, and combinations thereof.

24. The method of claim 1 wherein the multi-functional active hydrogen reactant having an equivalent weight less than 100 is a saccharide selected from the group consisting of glucose, fructose, sucrose, lactose, and combinations thereof.

25. The method of claim 1 wherein the multi-functional active hydrogen reactant having an equivalent weight less than 100 is a steroid.

26. The method of claim 1 wherein the multi-functional active hydrogen reactant having an equivalent weight less than 100 is selected from the group consisting of ascorbic acid, gluconic acid, glucoronic acid, glucosamine, and combinations thereof.

27. The method of claim 1 wherein the multi-functional isocyanate component is selected from the group consisting of lysine diisocyanate, derivatives of lysine diisocyanate, lysine triisocyanate, derivatives of lysine triisocyanate, and combinations thereof.

28. The method of claim 1 wherein the tissue comprises soft tissue.

29. An adhesive comprising a moisture-curable, isocyanate-functional component prepared by reacting:
(a) a multi-functional isocyanate component having an average functionality of 2; and
(b) a multi-functional active hydrogen component having an average functionality of at least 3 that includes at least 30% by weight, based upon the total weight of the multi-functional active hydrogen component, of a multi-functional active hydrogen reactant having an equivalent weight less than 100,
wherein:
(i) the ratio R of active hydrogen groups to isocyanate groups is selected such that 0.5<R<0.9,
(ii) the multi-functional active hydrogen component is free of multi-functional active hydrogen reactants having main chain ether or ester linkages, and
(iii) the adhesive is bio-degradable and suitable for application to biological tissue.

30. An adhesive comprising a moisture-curable, isocyanate-functional component prepared by reacting:
(a) a multi-functional isocyanate component having an average functionality of 3; and
(b) a multi-functional active hydrogen component having an average functionality of at least 2 that includes at least 30% by weight, based upon the total weight of the multi-functional active hydrogen component, of a multi-functional active hydrogen reactant having an equivalent weight less than 100,
wherein:
(i) the ratio R of active hydrogen groups to isocyanate groups is selected such that 0.33<R<0.9,
(ii) the multi-functional active hydrogen component is free of multi-functional active hydrogen reactants having main chain ether or ester linkages, and
(iii) the adhesive is biodegradable and suitable for application to biological tissue.

31. A biodegradable moisture-curable, isocyanate-functional composition comprising:
(A) the reaction product of:
(a) a multi-functional isocyanate component; and
(b) a multi-functional active hydrogen component comprising at least 30% by weight, based upon the total weight of the multi-functional active hydrogen component, of a multi-functional active hydrogen reactant having an equivalent weight less than 100, and
(B) an agent selected from the group consisting of catalysts, latent hardening agents, rheology modifying agents, and combinations thereof.

32. A composition prepared by reacting:
(a) a multi-functional isocyanate component having an average functionality of h; and
(b) a multi-functional active hydrogen component having an average functionality of at least f and consisting essentially of multi-functional active hydrogen reactants having an equivalent weight less than 100,
wherein a ratio R of active hydrogen groups to isocyanate groups is selected such that 1/h<R<0.9.
